(19) United States
(12) Patent Application Publication
(10) Pub. No.: US 2001/0037598 A1
(43) Pub. Date: Nov. 8, 2001

(54) Process for producing cetane improvers from triglycerides

(76) Inventors: Galen J. Suppes, Lawrence, KS (US); Joseph A. Heppert, Lawrence, KS (US); Mark H. Mason JR., Lawrence, KS (US)

Correspondence Address: James H. Marsh, Jr., SHOOK, HARDY & BACON L.L.P., 1200 Main Street, Kansas City, MO, 64105-2118, US

(21) Appl. No.: 09/736,669

(22) Filed: Dec. 13, 2000

Related U.S. Application Data
(60) Provisional application No. 60/170,601, filed on Dec. 14, 1999.

(51) Int. Cl. C10L 1/22; C07C 205/00

(52) U.S. Cl. 44/405; 560/156

(57) ABSTRACT

Cetane improvers based on triglycerides and petroleum fractions are disclosed that are at least one half as effective as commercially sold cetane improvers. In each case, the cetane improvers are nitrates produced through the nitration of medium to long chain compounds containing a double bond. Applications include use with diesel and alcohol fuels intended for use in diesel engines. The nitrates have advantages due to their good performance relative to their nitrogen content. Observed properties of some products indicate they also have lubricity and/or detergency enhancing capabilities when used with diesel fuel.

### CROSS REFERENCE TO RELATED APPLICATION

Priority pursuant to 35 U.S.C. § 119(e) is claimed from Provisional Application Ser. No. 60/170,601, filed Dec. 14, 1999.

### BACKGROUND OF THE INVENTION

1. Field of the Invention

The present invention relates generally to a method of producing cetane improvers intended for use with diesel fuels to improve fuel performance. More particularly, the present invention relates to a method of producing nitrates by nitration of triglycerides and to synergistic mixtures of these nitrates with other cetane improvers that both improve the cetane of a diesel fuel and improve the lubricity of the diesel fuel.

2. Description of the Prior Art

Nitrates of medium (5 to 8 carbon) to long (8 to 80 carbon) chain hydrocarbons are known to be effective as cetane improvers. This invention describes a novel process for producing these nitrates from triglyceride feedstocks. In related published art Poirier (U.S. Pat. No. 5,454,842) reports at least three different methods for producing nitrates from triglyceride feedstocks. The first of these includes steps of 1) hydrolysis of triglycerides to fatty acids, 2) esterification of the fatty acids with a diol such as ethylene glycol, and 3) nitration of the primary alcohol group of the glycol ester of the fatty acid. Nitration is performed using nitric acid in combination with another strong acid. This first method focuses on overall transesterification resulting in primary alcohols. Although not described in the patent, the long chain nitrates formed by this reaction had performance limitations related to the location of the ester bond between the nitrate groups and the major portion of the hydrocarbon chain. The ester tends to reduce the effectiveness of the free-radical decomposition that produces desired cetane improver performance. These nitrates are about one half as potent as compounds with nitrates located on the long hydrocarbon chain part of the molecule.

The second method described by Poirier includes 1) hydrolysis of triglycerides to fatty acids, 2) hydration of double bonds in the fatty acid chain via a reaction catalyzed by formic acid and reacting with hydrogen peroxide, and 3) nitration of the secondary alcohol groups. The resultant long chain nitrates had performance limitations due to limited solubility in diesel.

The third method described by Poirier includes 1) hydrolysis of triglycerides to fatty acids, 2) hydrogenation of the double bonds in the fatty acid chain, 3) esterification of the acids with methanol, 4) reduction of the ester bond to a primary alcohol, and finally 5) nitration of the alcohols. The process involves more steps than would be desired for a competitive production process. The performance was >60% that of medium to long chain alkyl nitrates. This third product was the basis of U.S. Pat. No. 4,454,842 with claims of good performance and high solubility.

Prior to the embodiments of this invention, the work of Poirier characterized the state of the art for producing cetane improvers from triglycerides. The processes of this invention improve upon Poirier's work for converting triglycerides into cetane improvers containing medium to long chain hydrocarbon groups and nitrates. Specific improvements include 1) direct nitration of double bonds to form nitrates with, 2) improved solubility (>0.5%) for these direct nitration products, and 3) improved performance by mixing nitrates produced from triglycerides with nitrates produced from polyglycols.

One aspect of this invention relates to a synergy resulting from mixing two cetane improvers. The performance of the mixture is better than would be expected based on performance of similar concentrations of each cetane improver used separately. Several such synergistic combinations have been reported in the literature, including U.S. Pat. Nos. 4,623,362 and 4,448,587. Despite the commonalities of synergistic combinations of cetane improvers, not all mixtures of cetane improvers exhibit synergy. In some cases, the performance of a mixture of cetane improvers is worse than expected based on performances of the individual cetane improvers. The synergies exhibited by cetane improvers of this invention have not been previously reported in published literature. The said patents do not consider cetane improvers produced from soybean oil or soybean oil derivatives.

Cetane Number Performance

The performance of cetane improvers depends on a multitude of factors. Incremental improvements in performance diminish substantially with increasing application rates in diesel fuel. Subtle changes in diesel fuel composition can also have a dramatic impact on the performance of a cetane improver. In the U.S., diesel fuel has a minimum pipeline specification of a 40 cetane number. Typical values of cetane numbers are between 42 and 47. An effective cetane improver will increase the cetane number by about 2 at application rates of 250 ppm. ASTM standards require the use of a cetane engine to determine a fuel's cetane number; however, cetane numbers are known to correlate well with ignition delay times. For much of the work in this application, cetane improver effectiveness is measured in decrease in ignition delay time relative to the decrease in ignition delay time caused by 2-ethylhexyl nitrate when applied at the same rate.

Diesel Fuel Lubricity

Diesel fuel lubricity is a term used to characterize the ability of the diesel fuel to lubricate the close-tolerance moving parts that rely on diesel's lubricity to minimize maintenance-related wear. The technical community relies on multiple methods to evaluate fuel lubricity; these methods are the Munson Roller on Cylinder Lubricity Evaluator (M-ROCLE), the European High Frequency Reciprocating Rig (HFRR), and Scuffing Load Ball on Cylinder Lubricity Evaluator (SLBOCLE). Further details are available in SAE Papers 1999-01-3590 and 982567.

Munson and Hertz (SAE Paper 1999-01-3588) reported the performance of twelve different additives, none of the additives were described in detail. Three were described as fuel conditioners, one as a sulfur substitute, three as lubricity additives, one as a commercial biodiesel additive, two as biodiesel additive A, and two as biodiesel additive B. The industrially accepted definition of biodiesel is a methyl or ethyl ester of a fatty acid. This type of non-detailed description of fuel additives is fairly common.

The nitrate products of this paper were evaluated for cetane improving capabilities as well as for effectiveness as lubricity enhancers. Since these products are not commercially available, no data is available on their performance either as cetane enhancers or as lubricity enhancers. Based on our laboratory's synthesis of approximately 100 different nitrates of soybean oil derivatives, a multitude of physical properties have been observed, including formation of polymer products with essentially no solubility in diesel, products with high solids content and very limited solubility in diesel, and reddish products with very limited solubility in diesel. No data has been published on how the addition of nitrate groups impacts a molecule's ability to enhance lubricity in a diesel fuel-in fact, a variety or properties can be produced depending upon the details of the chemical synthesis.

The M-ROCLE method reports lubricity in lubricity numbers (LN). An acceptable LN is 1.0, below a value of 1.0 additives can be used to increase the LN. Munson et al (SAE Paper 1999-01-3590) report fatty acid methyl esters and fatty acid ethyl esters to be effective in increasing lubricity of low-LN fuels. Some of the data of their study is reported in the following table:

| Additive | Rate of Addition (vol %) | LN |
|---|---|---|
| None | 0.0 | 0.813 |
| Canola Methyl Ester | 0.1 | 1.047 |
| Canola Methyl Ester | 0.25 | 1.177 |
| Canola Methyl Ester | 0.5 | 1.228 |
| Canola Methyl Ester | 1.00 | 1.292 |
| Canola Oil Derivative | 0.1 | 1.095 |
| Canola Oil Derivative | 0.25 | 1.195 |
| Canola Oil Derivative | 0.5 | 1.285 |
| Canola Oil Derivative | 1.00 | 1.368 |

### SUMMARY OF INVENTION

The present invention is a process that overcomes problems outlined above through an improved nitration process which results in 1) fewer processing steps, 2) improved product solubility, and 3) improved performance relative to the nitrogen content of the product. Furthermore, a fuel additive composition is identified that improves both cetane number and lubricity; this mixture does not rely on the additive of lubricity enhancers beyond that provided by the nitrate product.

The products of this invention may be used with diesel, ethanol, or related fuels which are used in compression-ignition engines. The products decrease ignition delay times and result in performance advantages associated with reduced emissions and easier cold-start.

An advantage of using triglycerides as feedstocks for production of cetane improvers is that triglycerides are produced from vegetation or livestock, and so, triglycerides are renewable feedstocks. An additional advantage of using triglycerides as feedstocks are that they are relatively low cost as compared to typical prices for nitrate-based cetane improvers.

To alleviate the problems of the prior art, the present invention provides a cetane improver for diesel fuel comprising a nitrated C₁-C₄ ester of a fatty acid. The ester is derived from a naturally occurring triglyceride and the nitration is accomplished by hydration of at least one double bond of the fatty acid and subsequent nitration of the secondary alcohols thus produced. In accordance with the concepts and principles of the invention, the cetane improver may also be a lubricity enhancer and/or a detergency enhancer. The invention also provides an improved diesel fuel comprising a cetane improver as described above.

In accordance with a preferred embodiment of the invention, the nitrated ester cetane improver of the invention may be formed by a process that includes transesterification of a vegetable oil. In another preferred embodiment of the invention, the nitrated ester is formed by a process that includes hydrolysis of a vegetable oil followed by esterification of fatty acids formed by such hydrolysis.

In further accord with the principles and concepts of the invention, an additive for a diesel fuel is provided whereby the cetane and the lubricity of the fuel is enhanced. The additive comprises a nitrated C₁-C₄ ester of a fatty acid as described above. When added to the diesel fuel in an effective amount, the additive is capable, of providing more than 90% of the cetane enhancement and more than 50% of the lubricity enhancement of said diesel fuel. The additive is also capable of providing more than 50% of the detergency enhancement of said diesel fuel.

The invention also provides a method for improving the cetane, lubricity and/or detergency of a diesel fuel. The method comprises admixing a nitrated C₁-C₄ ester of a fatty acid as described above with the diesel fuel. In one preferred aspect of the invention, the nitrated ester is formed by a process that includes transesterification of a vegetable oil. In yet another preferred aspect of the invention, the nitrated ester is formed by a process that includes hydrolysis of a vegetable oil followed by esterification of fatty acids formed by said hydrolysis.

The present invention also provides a method for improving the cetane, lubricity and/or detergency of a diesel fuel. The method comprises preparing a nitrated C₁-C₄ ester of a fatty acid by a process that includes converting a naturally occurring triglyceride into a fatty acid, hydrating at least one double bond of the fatty acid to produce a pair of secondary alcohol moieties on opposite sides of the hydrated double bond, and nitrating the secondary alcohols. The method further includes adding the nitrated ester to the diesel fuel. In a preferred aspect of the invention, the conversion of the triglyceride into a fatty acid includes transesterification of a vegetable oil. In another preferred aspect of the invention, the conversion of the triglyceride into a fatty acid includes hydrolysis of a vegetable oil followed by esterification of the fatty acids formed during hydrolysis.

### DETAILS OF THE PREFERRED EMBODIMENTS

This invention is a process for producing nitrates. Since this process 1) uses fewer steps during synthesis, 2) is effective with a variety of feedstocks including triglycerides, and 3) has a good performance relative to nitrogen content, this invention has advantages over alternatives. The molecular compounds of this invention provide both lubricating and cetane improving capabilities; other known compounds are only effective for lubricating or cetane improving purposes in fuels.

Feedstocks

Feedstocks for this process are limited to mixtures having average carbon numbers greater than 10 and having fatty acids or fatty acid derivatives in some of the components of the mixture. Direct or indirect nitration of the carbon-carbon double bonds is performed to synthesize the desired nitrates.

Direct nitration of the carbon-carbon double bonds is achieved by contacting the feedstock with dinitrogen pentoxide (N₂O₅) at temperatures between −40 and +50° C. and preferably between −20 and +30° C. The most preferred reaction temperatures are between −10 and +20° C. Minimum required contact time is typically less than 30 minutes after complete mixing. Appropriate precautions should be taken during mixing-semi-batch processing times may be dominated by the time necessary for safe mixing within allowable temperature deviations. N₂O₅ is preferably synthesized on site by methods known in the science.

Alternatively, direct nitration of the carbon-carbon double bonds is achieved by contacting the feedstock with a mixture of nitric acid and acetic anhydride at temperatures between −40 and +80° C. Available data suggests that a mixture of nitric acid and acetic anhydride produces N₂O₅ and may be an indirect method of achieving the same chemistry as described above—both result in high yields of nitrate (—ONO₂) and nitro groups (—NO₂) in place of some of the double bonds.

Methods known in the science must be followed to mix the reactants at a rate no greater than the rate at which the heat of reaction can be removed while maintaining desired reaction temperatures. In practice this typically translates to the use of a semi-batch reactor or a flow reactor with properly designed heat removal and mixing means.

Possible feedstock mixtures that have double bonds and can be reacted include triglycerides, derivatives of triglycerides, petroleum fractions, synthetic crude oil, tall oil, and derivatives of waste polymers. Derivatives of triglycerides and waste polymers can be formed by a variety of processes including hydrolysis, glycolysis, esterification, and transesterification. The preferred feedstocks are triglycerides or derivatives of triglycerides. Preferred triglycerides are vegetable oils, pork fat, and beef tallow. Preferred vegetable oils are soybean oil, mustard oil, corn oil, and waste cooking oils. Illustrative Examples 1, 2, 3, and 8 describe products and product performances for products prepared from soybean oil and oleic acid. Oleic acid is a refined derivative of soybean oil.

Stoichiometry of Nitrating Agents

When nitrating with mixtures of nitric acid and acetic anhydride, the nitrate groups originate from the nitrogen in the nitric acid, and so, the extent of nitration can be limited by the molar stoichiometry of nitric acid used for nitration. Stoichiometries of 1:2.2:2.2 for [carbon-carbon double bonds]:[nitric acid]:[acetic anhydride] result in near complete nitration; however, the embodiments of this invention are not limited to this stoichiometry.

Similarly, a 1:1.1 stoichiometry of [carbon-carbon double bonds]:[N₂O₅] results in near complete nitration; however, the embodiments of this invention are not limited to this stoichiometry.

Finishing methods known in the art can be used to remove excess acid, electrolytes, and volatile components from the product. The unreacted acids are preferably removed prior to use of the cetane improver. Finishing methods include but are not limited to extraction and vacuum distillation.

Product Solubility

Preferably, nitrates used as cetane improvers should be totally soluble in the fuel of application. Solubility problems typically do not occur with ethanol; however, for the preferred diesel applications, high degrees of nitration combined with ester or ether bonds within the nitrate compound can lead to solubility problems (i.e., solubilities less than 0.1%). Although typical application rates of cetane improvers in diesel are <0.1%, solubilities >0.2% are desired to reduce potential problems with mixing at industrial scales.

In particular, solubility problems can occur when both the average number of nitrate groups exceeds one nitrate group per 14 carbons and the average number of ester groups exceeds one ester group per 14 carbons. Preferably, the [total nitrogen content]:[carbon] ratio should be less than 1:24 (atom ration) when ester groups are present. The nitrogen may be present as both nitrate and nitrous groups with the nitrate groups providing the desired performance as a cetane improver.

The preferred method to limit nitration of the feedstock is to limit the stoichiometry of the nitrogen containing reactant. This approach typically only is effective when the nitrogen containing reactant or reactant mixture is added to a bulk continuous phase consisting primarily of the feedstock being nitrated. Preferred embodiments of this invention have solubilities >0.5%. These solubilities are achieved by limiting the stoichiometry of the nitrogen containing reactant and by adding the nitrogen containing reactant to the continuous feedstock phase. The embodiments of this invention include using this approach with direct nitration methods as well as indirect nitration methods including the second method of Poirier as described in the Description of Prior Art.

Multiple functionality

Preferred compositions and embodiments of this invention include nitrates of fatty acids and fatty acid derivatives that perform multiple value-added functions in a diesel fuel. Improving the cetane number is one of these functions. Additional value-added functions potentially include improving lubricity and detergent benefits.

Chemical compounds having both polarity and average straight-chain carbon numbers greater than 12 and preferably greater than 16 are known to have utility for enhancing lubricity of diesel fuels. Typical application rates are <1000 ppm; however, higher application rates do not typically have technical limitations.

Chemical structures representing methyl oleate (MO), a model compound for biodiesel, methyl oleate dinitrate (MODN), a model compound for biodiesel dinitrate, 2-ethylhexyl nitrate (EHN) and Ditertiary-Butyl peroxide (DTBP) are set forth below. MO is known to enhance lubricity. EHN and DTBP are known cetane improvers.

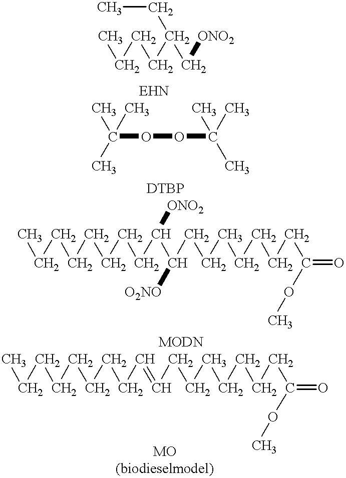

Fatty acid nitrates and nitrates of fatty acid derivatives have benefits associated with lubricity. Preferred applications include use of up to 1000 ppm with increases in lubricity of about 0.2 lubricity numbers per 1000 ppm additive.

The straight hydrocarbon chain with a polar or hydrogen bonding group on the end is also known to provide detergency. Preferred application rates are <1000 ppm.

Preferred means of using the multi-functionality of nitrates of fatty acid derivatives is as an additive package that enhances cetane number, lubricity, and detergency of a diesel fuel. Typically, additive packages designed to promote cetane number, lubricity, and detergency contain approximately equal masses of those constituents promoting these properties.

Within an additive package, the effectiveness of a component of that additive package is determined by changes in the desired property in the diesel fuel with and without the said component present in the additive package. For example, if a component of an additive package promotes cetane number and the boost in cetane number is 3 with the said component present while the boost in cetane number is 0.3 without the said component present, then the said component is considered to provide 90% of the cetane enhancement.

In the preferred additive packages of this invention more than 90% of the cetane enhancement is due to nitrates of fatty acid derivatives and more than 50% of the lubricity enhancement is due to the same nitrates of fatty acid derivatives. When the nitrate of the fatty acid derivative promotes detergency, the preferred fuel mixture embodiments of this invention have more than 50% of the detergency enhancement due to the nitrates of fatty acid derivatives.

Impact of Degree of Unsaturation

The nitrate product preferably flows without assistance and may either be a liquid or liquid containing suspended solids. The ASTM Standard D 2500 pour point temperature is preferably >15° C. Formation of a paste or solid with poor flow properties at 20° C. results when the average ratio of [carbon-carbon double bonds]:[carbon] in a triglyceride mixture feedstock is below 1:24. Preferably, the ratio of [carbon-carbon double bonds]:[carbon] is above 15. In practice, mixtures containing >45% soybean oil and <55% yellow grease (used triglyceride oils) provide a good balance of low cost (positive impact of yellow grease) and good flow properties (positive impact of soybean oil). Illustrative Example 5 provides observations on the impact of unsaturation on phase behavior.

The phase behavior of the nitrate products largely parallels the phase behavior of the reactants. The preferred means to obtain a product with desired phase behavior is to use triglyceride or triglyceride derivatives that are liquid at room temperature or to mix triglycerides that do not flow at room temperature with liquid triglycerides such that the resulting mixture flows at room temperature. Based on the data of illustrative example 5, liquid reactants and products typically include vegetable oils, fatty acids, or derivatives of fatty acids with a low degree of saturation in the carbon chain. Solid reactants and products tend to have average saturated fatty acid carbon numbers greater than 12 for the oils and greater than 14 for derivatives containing only one fatty acid.

Mixture Synergy

Mixtures of the nitrate of oleic acid ethylene glycol ester (OAEGN) with nitrates of di, tri, and tetra ethylene glycols were evaluated. These mixtures exhibited a synergy where combinations of the two classes of cetane exhibited a better performance than the use of a single class of cetane improver. In particular, a mixture of 0.2% diethyleneglycol dinitrate (DEGDN) with 0.8% OAEGN performed better than either 1% DEGDN or 1% OAEGN. DEGDN exhibited a better synergy than the other polyglycol nitrates. Illustrative Example 4 provides data on the synergy of or mixtures with DEGDN.

Biodiesel Nitrates

One embodiment of this invention improves the performance of biodiesel as a fuel additive. Biodiesel is typically a methyl or ethyl ester derivative of a triglyceride and typically contains an average of greater than 0.75 carbon-carbon double bonds per methyl or ethyl ester. Nitration methods described within this Summary of invention are effective for converting biodiesel into an effective cetane improver.

Performance Advantages

A preferred embodiment of this invention is the nitrate produced from contacting N₂O₅ with soybean oil at a stoichiometry most preferably between 0.6 and 1.1. The products provide a good cetane boost relative to the nitrogen content. As compared to 2-ethylhexyl nitrate which has a nitrogen to carbon ratio of 1:8, this soybean oil nitrate has a nitrogen to carbon ratio >1:20. Low nitrogen content is a performance advantage of this soybean oil nitrate.

Another advantage of triglyceride derived nitrates is that several triglyceride feedstocks are available that cost less than alcohol feedstocks used to produce 2-ethylhexyl nitrate.

While this invention has been described with special emphasis upon several preferred embodiments and in sufficient detail that a person knowledgeable in the science could perform the processes, it should be understood that within the scope of the appended claims the invention may be practiced otherwise than as specifically described herein. In particular, the following illustrative examples are described as batch or semi-batch practices-the embodiments of this invention also may be practiced as continuous processes or other processes consistent with the art and technology. Technology exists for the continuous synthesis and reaction of N₂O₅.

### ILLUSTRATIVE EXAMPLES

Preparation of Dinitrogen Pentoxide

Dinitrogen pentoxide (N₂O₅) was prepared and used for workups of several embodiments of this invention. It should be noted that N₂O₅ is a potentially hazardous material. It will react explosively with most organic materials. It is reported to explode when heated. It has a high vapor pressure, reacts with moisture in air to form nitric acid, and should not be inhaled. Recommended industrial practices include but are not limited to on-site production with immediate reaction, maintenance at temperatures <20° C., and operating guidelines that prevent the accumulation of more than a few grams of N₂O₅ as an intermediate.

The following method was used to prepare N₂O₅ for the embodiments of this invention; however, the embodiments of this invention are not limited to N₂O₅ chemistry or this method of preparation:

Dinitrogen pentoxide was prepared by a modification of literature procedures. Gaseous NO₂ (Aldrich) and an excess of O₃ were mixed and reacted immediately at room temperature in a 3-neck flask. (Careful attention should be paid to the gas leaving the reaction flask. It should be clear and colorless.) The gas stream flowed through a glass tube to a dry ice cold trap. After an appropriate period, the NO₂ lecture bottle was closed and the primary cold trap warmed to ambient temperature under an O₃ purge. The N₂O₅ sublimed to a cold finger and pure white crystals were collected and stored in a Schlenk tube packed in dry ice. 15-20 g of pure white powdery crystals are typically isolated.

### Illustrative Example 1

### Preparation and Performance of Soybean Oil Nitrate

An air-free cold addition funnel was connected to a 100 ml 3-neck flask, and to a Schlenk line. The air was evacuated and the apparatus re-filled with dry N₂. The cold trap on the addition funnel was then filled with dry ice. Under a nitrogen purge, 10 g of soybean oil (using an approx. MW of 880AMU this equals 1.13*10⁻² moles) previously dissolved in 30 ml of CH₂Cl₂ was added to the flask. The flask was immersed in an ice bath, and the oil solution, brought to 0° C. with constant stirring.

A capped airless addition tube was evacuated and re-filled with N₂. This was then tared on a top loading balance. Next, 1.2 g of N₂O (1.11*10⁻² mole) was added under strong N₂ purge and weighed on the same balance. The N₂O₅ was then added to the dropping funnel and dissolved in 50 ml of CH₂Cl₂. A strong flow of N₂ blown through an all glass pipette was used to agitate the N₂O₅. When these crystals were dissolved, the solution was added dropwise to the stirring oil solution. The addition usually took 2 hours, during which time the solution gradually became slightly darker. It changed from pale yellow, to light orange.

After the addition was complete the reaction was allowed to proceed for 1-2 hours at which time the solution was quenched with a slurry of ice water. This mixture was then neutralized with saturated NaHCO₃ and washed three times with distilled water. The methylene chloride was removed on a rotary evaporator, and the light orange oil was dissolved in dry diethyl ether (the ether was dried by distillation from sodium benzophenone). This solution was then dried with MgSO₄, filtered, rotary evaporated to dryness and the orange oil collected as a final product for use as a cetane improver.

Characterization and Analysis showed a yield >65%. ¹H NMR and IR analyses were performed to confirm conversion. Multiplets in the ¹H NMR spectra were at 0.87 ppm, 1.23 ppm, 1.60 ppm, 1.95 ppm, 2.25 ppm, 2.69 ppm, 4.25 ppm, 4.30 ppm, 5.3 ppm. Major IR peaks were seen at 3475 (small peak may be water), 2934, 2855, 1745, 1642, 1553, 1466, 1282, 1151, 968, 862, 714 cm⁻¹. The following peaks are not in the spectra of soy bean oil: 1642, 1553, 1378* (peak grows on the shoulder of an established peak), 1282, 863 cm⁻¹.

Decreases in ignition delay times are known to correlate with increases in cetane numbers. An isothermal laboratory combustor equipped with a diesel fuel injector and high-speed pressure transducer (Suppes et al, Industrial and Engineering Chemistry Research, Vol. 36, No. 10, pp. 4397-4404) was used to evaluate the performance of the product as a cetane improver. The performance of this product was compared to the performance of 2-ethylhexyl nitrate (EHN) in hexanes. Hexanes were used as the fuel for testing. The performance is reported in the following table where the decreases in ignition delay times caused by the product illustrate the product's effectiveness as a cetane improver.

### Illustrative Example 2

### Preparation and Performance of Oleic Acid Glycol Ester Nitrate

Using standard Schlenck techniques, an air-free cold addition funnel was connected to a 100 ml 3-neck flask. This was evacuated and re-filled with dry N₂. The trap on the funnel was filled with a mixture of crushed ice and salt water. Next, an airless addition tube was evacuated, filled with N₂, then weighed on a top loading balance. 4.5 g of N₂O₅ (0.04166 moles) was transferred to the airless addition tube and weighed. The N₂O₅ was transferred to a Schlenck flask and dissolved in 50 ml of cold CH₂Cl₂. When the powder dissolved, the solution was transferred by cannula to the air-free cold addition funnel.

12.5 g of oleic acid glycol ester (OAEG-OH) was prepared by known esterification methods with reactants oleic acid and ethylene glycol. The OAEG-OH (0.0383 moles) was dissolved in 20 ml of CH₂Cl₂ and transferred to the 3-neck flask. The flask was lowered into a 20° C. water bath. The nitrating solution was slowly added dropwise over the next 3 hours.

After the reaction was nearly complete (as judged by taking a small aliquot for ¹H NMR) the solution was quenched with an ice water slurry and neutralized with NaHCO₃(aq) then washed 3 times with distilled H₂O. This was dried over MgSO₄, filtered, the solvent removed in vacuo, and collected as an orange oil. Yields of >90% light orange to dark yellow oil are achievable. >90% conversion was confirmed by NMR analysis.

1H NMR and IR analyses were performed to confirm conversion. Major IR peaks (spectra were obtained on a Nicolet FT-IR spectrometer) taken on either NaCl plates or Teflon tape zeroed as background included 3470, 2934, 2846, 1754, 1650*, 1553**, 1460, 1375**, 1282*, 854* cm⁻¹ where * indicates —ONO₂ peaks (nitrate) and ** indicates —NO₂ peaks (nitro).)

Major ¹H NMR peaks (spectra obtained on either Bruker AM 500 MHZ or a Bruker DRX 400 MHZ instruments) for spectra done in CDCl₃ included multiplet@0.901 ppm, multiplet@1.28 ppm, multiplet@1.64 ppm, multiplet@2.1 ppm, multiplet@2.35, multiplet, singlet@4.27 (due to ethylene glycol dioleate)@4.36, multiplet@4.68, and multiplet@5.35 ppm.

The performance of this product (OAEGN) was compared to the performance of 2-ethylhexyl nitrate (EHN) in a test fuel (Test Fuel 2) comprised of 90% hexanes and 10% ethanol by mass. The performance is reported in the following table where the decreases in ignition delay times caused by the product illustrate the product's effectiveness as a cetane improver. Qualitative performances within each data series can be used to compare performances; however, data between the data sets does not provide valuable information since subtle changes in the injector, temperature, calibration, and vessel can impact data over long periods of time. The data shows OAEGN is an effective cetane improver.

### Illustrative Example 3

### Preparation and Performance of Oleic Acid Glycol Ester Nitrate

The oleic acid glycol ester nitrate of Illustrative Example 2 was prepared by dissolving 15 g of the oleic acid glycol ester with 15 g of acetic anhydride, then dropping 3.2 g of 90% HNO₃ into the oil solution over a period of 3-4 hrs at room temp. NMR analysis showed this product to be similar to the product of Illustrative Example 2.

### Illustrative Example 4

### Synergy of Mixture of 20% Diethyleneglycol Dinitrate (DEGDN) and 80% OAEGN

The data of Illustrative Example 2 compares the performance of 1% DEGDN, OAEGN, 50:50 mass ratio mixture of OAEGN and DEGDN, and an 80:20 mixture of OAEGN and DEGDN. Mixtures of the two nitrates performed better than either nitrate used by itself. This synergy appeared to be optimal at a ratio 80:20 OAEGN to DEGDN.

### Illustrative Example 5

### Impact of Degree of Unsaturation

Glycol ester nitrates of several fatty acids were prepared in a manner similar to that described by Illustrative Example 3. The following table summarizes observations of these tests.

| Fatty Acid | Phase Behavior |
|---|---|
| C_{20:0} | Solid |
| C_{18:0} | Solid |
| C_{16:0} | 20%-50% Solid |
| C_{14:0} | 20%-50% Solid |
| C_{12:0} | Liquid |
| C_{20:1} | Liquid |
| C_{18.1} | Liquid |
| C_{16.1} | Liquid |

Similar synthesis using yellow grease (waste vegetable oils) having an average degree of unsaturation of about 2 carbon-carbon double bonds per molecule resulted formation of a viscous paste. Use of soybean oil with an average degree of unsaturation of >3 carbon-carbon double bonds per molecule resulted in formation of a liquid product. Use of a 50:50 mixture of soybean oil and yellow grease resulted in a product with some entrained solids but flowed well at room temperature. Combined, these observations illustrate that unsaturation promotes liquid phase behavior at room temperature for typical nitrates of vegetable oil derivatives.

While this invention has been described with special emphasis upon several preferred embodiments and in sufficient detail that a person knowledgeable in the science could perform the processes, it should be understood that within the scope of the appended claims the invention may be practiced otherwise than as specifically described herein.

### Illustrative Example 6

### Preparation and Performance of Biodiesel Polynitrate from Dinitrogen Pentoxide

Biodiesel (the simple alcohol esters of fatty acids prepared by transesterification of vegetable oil with a simple alcohol like methanol, ethanol etc) was epoxidized with an excess 50% hydrogen peroxide using 98% formic acid as a catalyst. The resulting epoxides were extracted into ether, neutralized, washed, and isolated in vacuo Using standard Schlenck techniques, an air-free cold addition funnel was connected to a 100 ml 3-neck flask. This was evacuated and re-filled with dry N₂. The trap on the funnel was filled with a mixture of crushed ice and salt water. Next, an airless addition tube was evacuated, refilled with N₂, then weighed on a top loading balance. Next, 6.8 g (0.063 moles) of N₂O₅ washed weighed into an air-free addition tube under strong nitrogen purge. That was dissolved in 30 ml of CH₂Cl₂ previously cooled to 0° C. Twenty grams (0.064 moles) of epoxy-biodiesel was dissolved in 25 ml of CH₂Cl₂ and slowly added to the N₂O₅ solution. The rate of addition was adjusted to maintain the reaction temperature below +5° C. After the addition was complete the mixture was allowed to stir for 30-45 minutes. Then the solution was quenched in an ice water slurry, extracted into diethyl ether, neutralized, and isolated in vacuo. Products were characterized by FT-IR spectroscopy. FT-IR data: peaks@2934, 2864, 1745, 1645, 1553 (low intensity peak), 1439, 1274, 860 cm⁻¹.

| | | Ignition Delay Time | | | ΔI.D.T.Expressed as | | |
|---|---|---|---|---|---|---|---|
| Biodiesel polynitrate | Test | Expressed in ms: | | | % ΔI.D.T. of EHN: | | |
| verses EHN: | Date | 750K | 800K | 850K | 750K | 800K | 850K |
| Hexanes | 8/99 | 23.02 | 15.27 | 8.33 | 0 | 0 | 0 |
| Hexanes + 0.5% | 8/99 | 15.80 | 11.37 | 6.21 | 113 | 62 | 65 |
| Bk2P291 | | | | | | | |
| Hexanes + 0.5% EHN | 8/99 | 16.66 | 8.99 | 5.06 | 100 | 100 | 100 |

### Illustrative Example 7

### Preparation and Performance of Biodiesel Polynitrate from Nitric Acid Mixtures

Biodiesel (the simple alcohol esters of fatty acids prepared by transesterification of vegetable oil with a simple alcohol like methanol, ethanol etc) was epoxidized with an excess 50% hydrogen peroxide using 98% formic acid as a catalyst. The resulting epoxides were extracted into diethyl ether, neutralized, washed, and isolated in vacuo. The resulting epoxides were subject to ring opening hydrolysis in water with catalytic amounts of sulfuric acid. The resulting fatty polyols were extracted into diethyl ether, neutralized, and isolated in vacuo.

In a three-neck 100 ml flask, equipped with a dropping funnel and stirrer, 7.5 ml (0.166 moles) of 90% nitric acid was cooled to 0° C. and mixed with 9.0 ml of 18M H₂SO₄. This mixture was was cooled to 0° C. and mixed with 1 mg of urea. In a separate flask 25 g (≈0.063 moles) of hydroxy-biodiesel added to the addition funnel. The oil was dropped into the acid over the next two hours. Careful control of stirring and cooling was used to maintain the temperature below +10° C., but above 0° C. After the addition was complete the solution was allowed to stir for 30-45 minutes. After that period the mixture was quenched by pouring it in ice water, extracted into ethyl ether, neutralized with NaHCO₃, and washed twice more. The ether was removed in vacuo, and FT-IR and ¹HNMR spectroscopy was used to analyze the product oil. ¹H NMR data: small multiplet@5.2 ppm, sharp intense singlet@3.68 ppm, triplet@2.24 ppm, multiplets@1.7, 1.38, and 0.89 ppm. FT-IR data: peaks@2934, 2864, 1745, 1645, 1553 (low intensity peak), 1439, 1274, 860 cm⁻¹.

| | | Ignition Delay Time | | | ΔI.D.T.Expressed as | | |
|---|---|---|---|---|---|---|---|
| Biodiesel polynitrate | Test | Expressed in ms: | | | % ΔI.D.T. of EHN: | | |
| verses EHN: | Date | 750K | 800K | 850K | 750K | 800K | 850K |
| Hexanes (base fuel) | 8/99 | 24.77 | 16.63 | 9.24 | 0 | 0 | 0 |
| Hexanes + 0.5% | 8/99 | 21.06 | 12.89 | 7.06 | 67 | 88 | 71 |
| Bk4p167* | | | | | | | |
| Hexanes + 0.5% EHN | 8/99 | 19.24 | 12.40 | 6.15 | 100 | 100 | 100 |
| Hexanes | 10/99 | 24.1 | 16.6 | 8.6 | 0 | 0 | 0 |
| Hexanes + 0.5% | 10/99 | 19.7 | 14.1 | 6.8 | 78 | 60 | 75 |
| Bk4p237* | | | | | | | |
| Hexanes + 0.5% EHN | 10/99 | 18.5 | 12.4 | 6.2 | 100 | 100 | 100 |

*Bk4p167 was synthesized with a mixture of nitric and sulfuric acids; Bkp237 was made with acetic anhydride and nitric acid.

| | | | Cetane Engine Results: | | | | |
|---|---|---|---|---|---|---|---|
| Biodiesel | | Application | | Blending | ΔI.D.T.Expressed as | | |
| polynitrate | Test | Rate in | Δ Cetane | cetane | % ΔI.D.T. of EHN: | | |
| verses EHN | Date | ppm | Number | number | 750K | 800K | 850K |
| Bk2p291 | 7/99 | 1000 | 3.1 | 3142 | 113 | 62 | 65 |
| MC0400* | 7/99 | 1000 | 2.8 | 2843 | na | na | na |
| Bk4pl67 | 8/99 | 1000 | 3.0 | 3044 | 67 | 88 | 71 |
| | | 250 | 2.2 | 8844 | 67 | 88 | 71 |
| MCC400* | 8/99 | 1000 | 3.9 | 3944 | na | na | na |
| Bk4p237 | 11/99 | 1000 | 1.1 | 1146 | 78 | 60 | 75 |
| Neat EHN | 11/99 | 1000 | 2.7 | 2746 | 100 | 100 | 100 |

MCC400 is a commercial blend of diesel fuel additives which contains EHN.
*The application rate of MCC400 was adjusted to give 1000 ppm EHN.

| | | Ignition Delay Time | | | ΔI.D.T.Expressed as | | |
|---|---|---|---|---|---|---|---|
| Soybean | Test | Expressed in ms: | | | % ΔI.D.T. of EHN: | | |
| polynitrate: | Date | 750K | 800K | 850K | 750K | 800K | 850K |
| Hexanes (base fuel) | 8/98 | 18.58 | 6.93 | 5.07 | 0 | 0 | 0 |
| 0.5% MMBk2P267 | 8/98 | 14.98 | 5.62 | 4.44 | 56 | 77 | 50 |
| 0.5% EHN | 8/98 | 12.11 | 5.23 | 3.82 | 100 | 100 | 100 |

### Illustrative Example 8

### Preparation and Performance of Triglyceride Polynitrate from Nitric Acid Mixtures

The same method applied to fatty acid methyl esters may also be applied to triglycerides The advantage of the triglycerides are economic as they cost less than biodiesel. Though the biodiesel polynitrates show greater activity.

### Illustrative Example 9

### Preparation and Performance of Vicinal Ether-Nitrate Fatty Esters.

Preparation of Methoxy-Hydroxy-Biodiesel:

A 500 ml two neck round bottom flask was equipped with a condenser, a thermometer and stirrer. To this flask 55 g (1.72 moles) of anhydrous methanol was added and mixed with 15 g (0.048 moles) of epoxy-biodiesel. Next, 0.5 ml of 18M H₂SO₄ was dissolved in 5 ml of methanol and that acidic solution was rapidly added to the homogenous methanol/oil mixture. The mixture was heated to 40° C. for 17 hrs. The reaction was extracted into ether and neutralized with NaHCO₃. The ether was removed in vacuo giving an 85% yield. ¹H NMR data: sharp intense singlet@3.68 ppm, a series of low intensity singlets which range from 3.43 to 3.51 ppm, a triplet@2.34 ppm, multiplets@1.65, 1.33, 1.27, 1.23 and 0.89 ppm. FT-IR data: broad peak@3466, 2933, 2872, 1736, 1473, 1203, 1107, 723 cm⁻¹.

Preparation of methoxy-biodiesel nitrate (MEN):

The nitration apparatus used for this preparation is the same as the one used to nitrate hydroxy-biodiesel discussed above. Five grams (0.014 moles) of methoxy-hydroxy-biodiesel was mixed with 3 ml of acetic anhydride then dropped into 2.4 ml (0.58 moles) of HNO₃. Again the rate of addition and stirring were adjusted to keep the temperature between 0° C. and +10° C. The reaction was quenched and worked up in the same fashion as the other nitrations. Product yields of up to 70% are possible. NMR data: sharp intense singlet@3.68 ppm, a series of low intensity singlets which range from 3.43 to 3.51 ppm, a triplet@2.34 ppm, multiplets@1.65, 1.4, 1.27, 1.23 and 0.89 ppm. FT-IR data: large, intense peaks were seen@2925, 2864, 1736, 16, 32, 1473, 1282, 864 cm⁻¹.

The same method may also be applied to vegetable oil triglycerides. First, the oil is epoxidized, then the epoxides are opened in an excess of alcohol, and the resultant vicinal ether-hydroxy fats are nitrated with nitric acid mixtures.

| | | Ignition Delay Time | | | ΔI.D.T.Expressed as | | |
|---|---|---|---|---|---|---|---|
| | Test | Expressed in ms: | | | % ΔI.D.T. of EHN: | | |
| Compound | Date | 750K | 800K | 850K | 750K | 800K | 850K |
| Hexanes | 5/3/99 | 28.68 | 18.54 | 9.58 | 0 | 0 | 0 |
| 0.5% Bk3P227 | 5/3/99 | 19.31 | 10.45 | 5.76 | 83 | 95 | 104 |
| (MBN) | | | | | | | |
| 0.5% EHN | 5/3/99 | 17.43 | 10.00 | 5.93 | 100 | 100 | 100 |
| Hexanes | 5/19/99 | 29.04 | 17.97 | 9.55 | 0 | 0 | 0 |
| 0.5% Bk3p293 | 5/19/99 | 17.69 | 10.90 | 5.97 | 101 | 88 | 97 |
| (MBN) | | | | | | | |
| 0.5% EHN | 5/19/99 | 17.84 | 9.99 | 5.85 | 100 | 100 | 100 |
| Hexanes | 5/19/99 | 29.04 | 17.97 | 9.55 | 0 | 0 | 0 |
| 0.5% Bk3p301 | 5/19/99 | 23.03 | 12.48 | 6.00 | 53 | 69 | 91 |
| (ESN) | | | | | | | |
| EHN | 5/19/99 | 17.84 | 9.99 | 5.85 | 100 | 100 | 100 |
| Hexanes | 5/11/99 | 27.14 | 16.86 | 9.95 | 0 | 0 | 0 |
| 0.5% Bk3p265 | 5/11/99 | 21.4 | 12.35 | 6.34 | 73 | 60 | 72 |
| (MSN) | | | | | | | |
| 0.5% Bk3p269 | 5/11/99 | 23.23 | 12.29 | 6.68 | 50 | 60 | 65 |
| (MSN) | | | | | | | |
| EHN | 5/11/99 | 19.28 | 9.31 | 4.97 | 100 | 100 | 100 |

MBN = methoxy-biodieselnitrate;
ESN = ethoxy-soybean oil nitrate;
MSN = methoxy-soybean oil nitrate.

### Illustrative Example 10

### Preparation and Performance of Biodiesel Polynitrate from Nitric Acid Mixtures at 350 ml Scale.

The following recipe was followed to prepare 350 ml of biodiesel polynitrate in a 1 liter laboratory semi-batch reactor.

| | Time |
|---|---|
| Perform Epoxidation Reaction | |
| Reactor empty | |
| Set temperature to 30° C. | |
| Add 688 ml biodiesel | |
| Start stirring | |
| Add 59 ml 97% formic acid | 5 min |
| Slowly add 240 ml 50% hydrogen peroxide | 1 hr |
| Allow to React | ˜10 hrs |
| Perform Hydrolysis Reaction | |
| Increase temperature setting to 85° C. | |
| Add 500 ml water | |
| When temperature reaches 50-55° C. | |
| Add 25 ml 18M sulfuric acid in 400 ml watermin | |
| Let react with strong agitation | 4 hrs |
| Quench reaction with 400 ml water | |
| Discontinue stirring | |
| Allow to separate | |
| Remove aqueous layer | |
| Wash twice with water | |
| Remove polyhydroxy biodiesel from reactor | 20 min |

Perform Nitration (only 350 ml of the polyhydroxy is used due to
reactor size)

| | |
|---|---|
| Add 250 ml 90% nitric acid to empty reactor | |
| Slowly add 312 ml 18M sulfuric acid | |
| Allow to cool to 10° C. | 15-20 min |
| With excellent stirring slowly add 350 ml polyhydroxy | 1 hr |
| biodiesel, keeping the temperature below 15° C. | |
| Slowly add ice to quench the reaction, the equivalent to | 30 min |
| 500-700 ml of water, wash three times with water | |
| Dry the oil under vacuum at 40-50° C. | 30 min |
| Total time | 16-18 hrs |

This recipe resulted in a product with a yellow, slightly greenish tint. The products of examples 1 through 10 resulted in products with a reddish tint.

Ignition delay times were evaluated in a constant volume combustor wherein the performance product was compared to the performance of EHN tested at the same concentration. The testing results are summarized in the table below.

IGNITION DELAY TIMES IN ms

| | 0.1% Add | | 0.2% Add | | 0.4% Add | | Hex- |
|---|---|---|---|---|---|---|---|
| T (K) | BK1P41 | EHN | BK1P41 | EHN | BK1P41 | EHN | anes |
| 750 | 15.9 | 16.8 | 15.9 | 16.8 | 16.9 | 19.0 | 40.1 |
| 800 | 12.6 | 14.1 | 12.6 | 14.1 | 12.4 | 12.3 | 20.5 |
| 850 | 7.9 | 7.6 | 7.9 | 7.6 | 7.4 | 6.6 | 13.6 |

STANDARD DEVIATIONS ms

| | | | | | | | |
|---|---|---|---|---|---|---|---|
| 750 | 1.05 | 1.43 | 1.12 | 1.56 | 1.36 | 1.79 | 1.42 |
| 800 | 1.48 | 1.67 | 1.41 | 1.16 | 1.15 | 1.35 | 1.38 |
| 850 | 0.81 | 1.45 | 1.08 | 0.61 | 0.95 | 0.94 | 1.43 |

In addition to direct measurement of ignition delay times, this product was also evaluated in a cetane engine at 250 ppm. The table below summarizes the results of these tests.

| | Cetane |
|---|---|
| Fuel | Number |
| Test Diesel | 37.3 |
| Test Diesel + 250 ppm BK1P41 | 38.2 |
| Test Diesel + 250 ppm EHN | 40.3 |

### Illustrative Example 11

### Performance of Nitrates of Fatty Acid Derivatives.

The product of illustrative example 10 is a largely clear product with a yellowish-green tint. The freezing point of the liquid is below −10° C. and it adheres to glassware surfaces similar to soybean oil and biodiesel. Based on these qualitative observations, the liquid was estimated to have lubricating characteristics.

We claim:

1. A cetane improver for diesel fuel comprising a nitrated C₁-C₄ ester of a fatty acid, said ester being derived from a naturally occurring triglyceride, said nitration having been accomplished by hydration of at least one double bond of said fatty acid and subsequent nitration of the secondary alcohols thus produced.

2. A cetane improver as set forth in claim 1 which is a lubricity enhancer.

3. A cetane improver as set forth in claim 1 which is a detergency enhancer.

4. A cetane improver as set forth in claim 2 which is a detergency enhancer.

5. An improved diesel fuel comprising the cetane improver of claim 1, 2, 3 or 4.

6. A cetane improver as set forth in claim 1, 2, 3 or 4 where said ester is formed by a process including transesterification of a vegetable oil.

7. A cetane improver as set forth in claim 1, 2, 3 or 4 where said ester is formed by a process including hydrolysis of a vegetable oil followed by esterification of fatty acids formed by said hydrolysis.

8. An additive for providing a diesel fuel with enhanced cetane and enhanced lubricity comprising a nitrated C₁-C₄ ester of a fatty acid as set forth in claim 2, said additive being capable, when added to said diesel fuel in an effective amount, of providing more than 90% of the cetane enhancement and more than 50% of the lubricity enhancement of said diesel fuel.

9. An additive for providing a diesel fuel with enhanced cetane, enhanced lubricity and enhanced detergency comprising a nitrated C₁-C₄ ester of a fatty acid as set forth in claim 4, said additive being capable, when added to said diesel fuel in an effective amount, of providing more than 90% of the cetane enhancement, more than 50% of the lubricity enhancement of said diesel fuel and more than 50% of the detergency enhancement of said diesel fuel.

10. An additive for providing a diesel fuel with enhanced cetane and enhanced detergency comprising a nitrated C₁-C₄ ester of a fatty acid as set forth in claim 3, said additive being capable, when added to said diesel fuel in an effective amount, of providing more than 90% of the cetane enhancement and more than 50% of the detergency enhancement of said diesel fuel.

11. A method for improving the cetane of a diesel fuel comprising admixing a nitrated C₁-C₄ ester of a fatty acid as set forth in claim 1 with said diesel fuel.

12. A method for improving the cetane and the lubricity of a diesel fuel comprising admixing a nitrated C₁-C₄ ester of a fatty acid as set forth in claim 2 with said diesel fuel.

13. A method for improving the cetane and the detergency of a diesel fuel comprising admixing a nitrated C₁-C₄ ester of a fatty acid as set forth in claim 3 with said diesel fuel.

14. A method for improving the cetane, the detergency and the lubricity of a diesel fuel comprising admixing a nitrated C₁-C₄ ester of a fatty acid as set forth in claim 4 with said diesel fuel.

15. A method as set forth in claim 11, 12, 13 or 14 wherein said nitrated ester is formed by a process including transesterification of a vegetable oil

16. A method as set forth in claim 11, 12, 13 or 14 wherein said nitrated ester is formed by a process including hydrolysis of a vegetable oil followed by esterification of fatty acids formed by said hydrolysis.

17. A method for improving the cetane of a diesel fuel comprising:
preparing a nitrated C₁-C₄ ester of a fatty acid by a process that includes converting a naturally occurring tryglyceride into a fatty acid, hydrating at least one double bond of said fatty acid to produce a pair of secondary alcohol moieties on opposite sides of the hydrated double bond, and nitrating the secondary alcohols; and
adding said nitrated ester to said diesel fuel.

18. A method as set forth in claim 17, wherein said converting includes transesterification of a vegetable oil.

19. A method as set forth in claim 17, wherein said converting includes hydrolysis of a vegetable oil followed by esterification of the fatty acids formed during hydrolysis.

20. A method for improving the cetane and lubricity of a diesel fuel comprising:
preparing a nitrated C₁-C₄ ester of a fatty acid by a process that includes converting a naturally occurring tryglyceride into a fatty acid, hydrating at least one double bond of said fatty acid to produce a pair of secondary alcohol moieties on opposite sides of the hydrated double bond, and nitrating the secondary alcohols; and
adding said nitrated ester to said diesel fuel.

21. A method as set forth in claim 20, wherein said converting includes transesterification of a vegetable oil.

22. A method as set forth in claim 20 wherein said converting includes hydrolysis of a vegetable oil followed by esterification of the fatty acids formed during hydrolysis.

23. A method for improving the cetane and detergency of a diesel fuel comprising:
preparing a nitrated C₁-C₄ ester of a fatty acid by a process that includes converting a naturally occurring tryglyceride into a fatty acid, hydrating at least one double bond of said fatty acid to produce a pair of secondary alcohol moieties on opposite sides of the hydrated double bond, and nitrating the secondary alcohols; and
adding said nitrated ester to said diesel fuel.

24. A method as set forth in claim 23, wherein said converting includes transesterification of a vegetable oil.

25. A method as set forth in claim 23, wherein said converting includes hydrolysis of a vegetable oil followed by esterification of the fatty acids formed during hydrolysis.

26. A method for improving the cetane, lubricity and detergency of a diesel fuel comprising:
preparing a nitrated C₁-C₄ ester of a fatty acid by a process that includes converting a naturally occurring tryglyceride into a fatty acid, hydrating at least one double bond of said fatty acid to produce a pair of secondary alcohol moieties on opposite sides of the hydrated double bond, and nitrating the secondary alcohols; and
adding said nitrated ester to said diesel fuel.

27. A method as set forth in claim 26, wherein said converting includes transesterification of a vegetable oil.

28. A method as set forth in claim 26, wherein said converting includes hydrolysis of a vegetable oil followed by esterification of the fatty acids formed during hydrolysis.
